(19) United States
(12) Patent Application Publication
(10) Pub. No.: US 2025/0057432 A1
(43) Pub. Date: Feb. 20, 2025

(54) HEARTBEAT SENSOR DEVICE

(71) Applicants: Daniel Antwi - Amoabeng, Lake Charles, LA (US); David Rudd, Lake Charles, LA (US)

(72) Inventors: Daniel Antwi - Amoabeng, Lake Charles, LA (US); David Rudd, Lake Charles, LA (US)

(21) Appl. No.: 18/233,536

(22) Filed: Aug. 14, 2023

(51) Int. Cl. A61B 5/024; A61B 5/00

(52) U.S. Cl. CPC A61B 5/02427; A61B 5/6833; A61B 5/7445

(57) ABSTRACT

A heartbeat sensor device for visually indicating a pulse includes a housing attached to an adhesive band. A photoplethysmograph sensor is positioned within the housing and is electronically coupled to a central processing unit. The photoplethysmograph sensor measures the pulse. The central processing unit controls a pulse indication light, causing the pulse indication light to illuminate synchronously with the pulse measured by the photoplethysmograph sensor. The adhesive band sticks the heartbeat sensor device to the skin, for example over the carotid artery, where the photoplethysmograph sensor can detect light absorption changes in the skin caused by the pulse. Alternatively, the heartbeat sensor device may include a transducer connection port configured to receive a transducer cable. The transducer cable enables communication between the central processing unit and a display screen. In such embodiments, the display screen provides the visual pulse indicators.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to heartbeat sensors and more particularly pertains to a new heartbeat sensor for visually indicating a pulse.

### (2) Description of Related Art Including Information Disclosed Under 37 CFR 1.97 and 1.98

The prior art relates to heartbeat sensors. Some heartbeat sensors use photoplethysmography to measure the changes in light absorption of the skin caused by a pulse. Such sensors are commonly used in traditional pulse oximeters and have been adapted for use in smart watches, fitness trackers, and other wristbands that measure heart rate or oxygen saturation of the blood.

An accurate pulse check is vital for life-saving chest compressions in a patient suffering from cardiac arrest. Pulse check by carotid palpation has a relatively low accuracy rate of only about 65%. If the carotid artery is difficult to locate, or the pulse is hard to detect, that can cause a significant delay in the progression of the life-saving chest compressions. Devices such as a carotid point-of-care ultrasound can increase the accuracy rate of the pulse checks, but that approach relies on operator availability and competence as well as the ready availability of the ultrasound device at the scene of the resuscitation attempt. Such devices can also significantly increase the duration of pulse checks by adding additional steps, such as locating the device, preparing the device, and preparing the patient. Delays in finding and confirming a pulse rate cause delays in resuming chest compressions, which can significantly lower the chances that the patient survives cardiac arrest. Continuing chest compressions after the patient has a return of spontaneous circulation can also be dangerous, further increasing the importance of quick and accurate pulse checks. In most situations, the pulse check is a major decision branch-point that depends on the manual dexterity and subjective assessment of the healthcare professional attending to the patient.

Thus, there is a need in the art for a reliable and objective pulse check device that is fast and easy to use. There is also a need in the art for a device that provides real-time feedback on the presence of a self-sustaining pulse, which indicates the return of spontaneous circulation.

### BRIEF SUMMARY OF THE INVENTION

An embodiment of the disclosure meets the needs presented above by generally comprising a heartbeat sensor device that can be placed over an artery to visually indicate the presence and rate of a pulse. The heartbeat sensor device has a housing attached to an adhesive band. A photoplethysmograph sensor is positioned within the housing and is electronically coupled to a central processing unit. The photoplethysmograph sensor measures a pulse. The central processing unit controls a pulse indication light, causing the pulse indication light to illuminate synchronously with the pulse measured by the photoplethysmograph sensor. The adhesive band sticks the heartbeat sensor device to the skin, for example over the carotid artery, where the photoplethysmograph sensor can detect light absorption changes in the skin caused by the pulse. Alternatively, the heartbeat sensor device may include a transducer connection port that enables communication between the central processing unit and a display screen. In such embodiments, the display screen provides the visual pulse indicators.

There has thus been outlined, rather broadly, the more important features of the disclosure in order that the detailed description thereof that follows may be better understood, and in order that the present contribution to the art may be better appreciated. There are additional features of the disclosure that will be described hereinafter, and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front isometric view of a heartbeat sensor device according to an embodiment of the disclosure.

FIG. 2 is a bottom rear perspective view of an embodiment of the disclosure.

FIG. 3 is a bottom rear isometric view of an embodiment of the disclosure.

FIG. 4 is a front view of an embodiment of the disclosure.

FIG. 5 is an in-use view of an embodiment of the disclosure.

FIG. 6 is a top front isometric view of an embodiment of the disclosure.

FIG. 7 is a side view of an embodiment of the disclosure.

FIG. 8 is an in-use view of an embodiment of the disclosure.

### DETAILED DESCRIPTION OF THE INVENTION

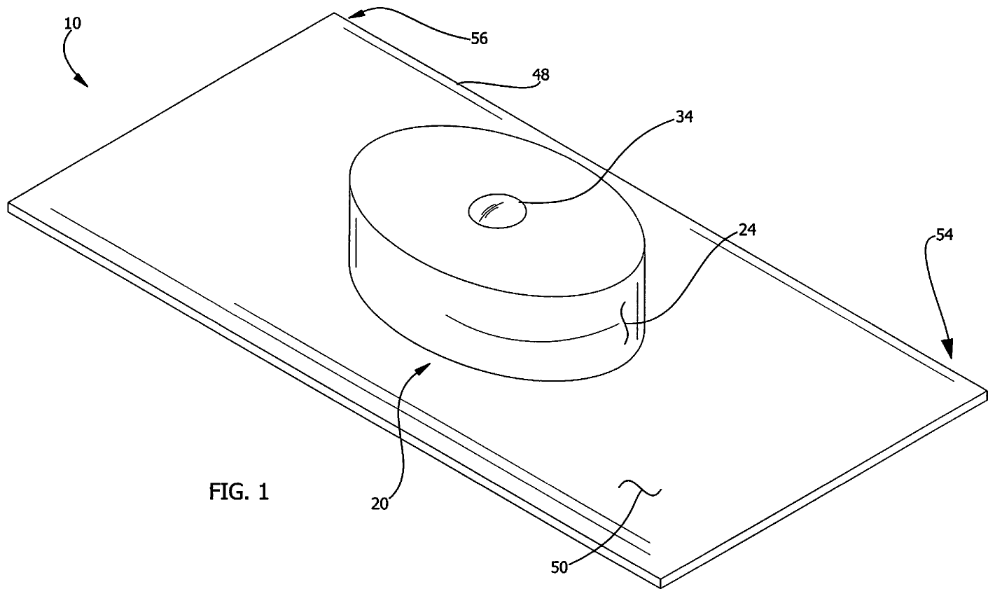

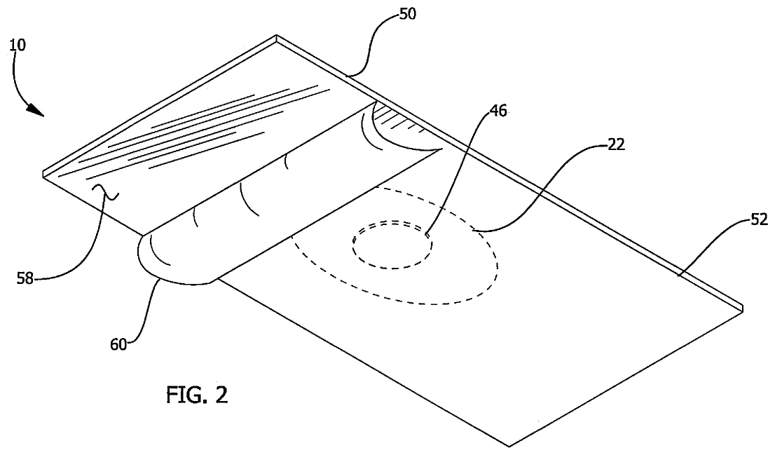

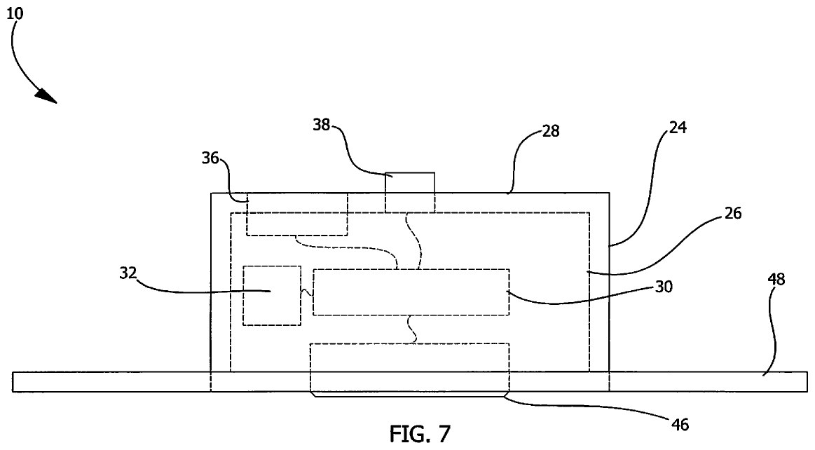

With reference now to the drawings, and in particular to FIGS. 1 through 8 thereof, a new heartbeat sensor embodying the principles and concepts of an embodiment of the disclosure and generally designated by the reference numeral 10 will be described.

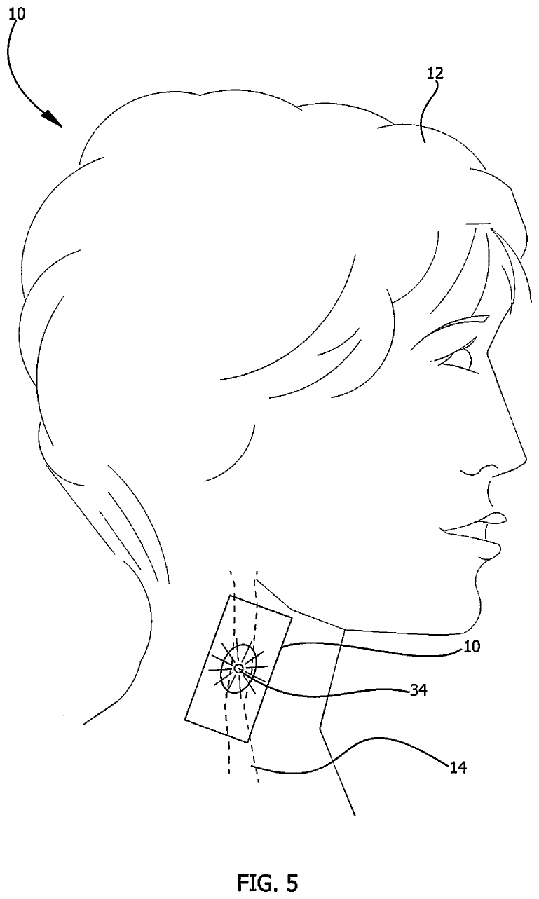

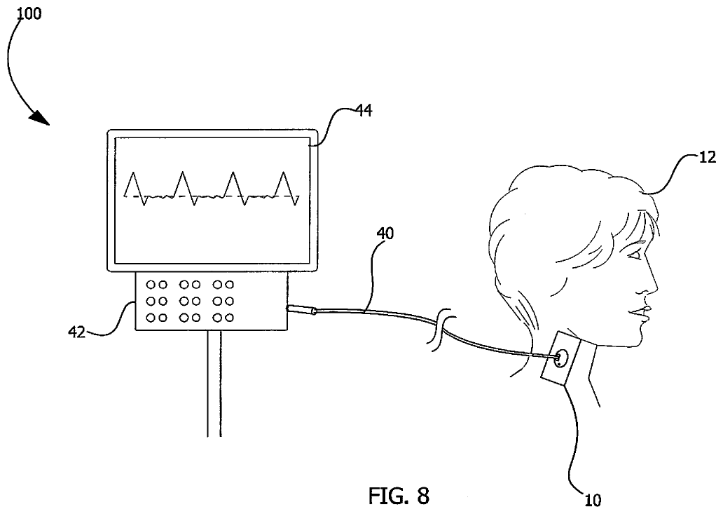

As best illustrated in FIGS. 1 through 8, the heartbeat sensor device 10 generally comprises a housing 20 having a bottom wall 22 and a perimeter wall 24 that is attached to and extends upwardly from the bottom wall 22. The perimeter 24 and bottom 22 walls bound an interior space 26. The perimeter wall may also include a top wall 28 that is opposite the bottom wall 22. In FIGS. 1-8, the top 28 and bottom 22 walls are generally shaped like an oval or an ellipse, but the housing 20 may have any shape. As shown in FIGS. 5 and 8, the heartbeat sensor device 10 is generally configured for placement over a carotid artery 14 of a patient 12. The heartbeat sensor device 10 may be placed over the right common carotid artery 14, so the housing 20 will generally be quite small. For example, the perimeter wall 24 may have a height ranging between 8 centimeters and 12 centimeters.

A central processing unit 30 may be positioned within the interior space 26. For example, the central processing unit 30 may be mounted to the housing 20. A battery 32 may also be positioned within the interior space 26, for example being mounted to the housing 20. The battery 32 is electronically coupled to the central processing unit 30. The battery 32 acts as a power source, providing energy for the central processing unit 30 and the other electrical components of the heartbeat sensor device 10.

A photoplethysmograph sensor 46 is positioned within the interior space 26. The photoplethysmograph sensor 46 may be mounted to the housing 20 and is generally directed outwardly away from the bottom wall 22. The photoplethysmograph sensor 46 is electronically coupled to the central processing unit 30. The photoplethysmography sensor 46 is configured to measure a pulse and transmit the measurement of the pulse to the central processing unit 30.

The embodiments depicted in FIGS. 1, 4, and 5 include a pulse indication light 34. The pulse indication light 34 is electronically coupled to the central processing unit 30. The pulse indication light 34 may be mounted to the housing 20, for example being directed outwardly away from the perimeter wall 24. In some embodiments, the pulse indication light may be positioned on the top wall 28. The pulse indication light 34 may be any type of light, such as a light emitting diode. The pulse indication light 34 is controlled by the central processing unit 30. The central processing unit 30 illuminates the pulse indication light 34 based on the measurement of the pulse provided by the photoplethysmograph sensor 46. For example, the central processing unit 30 may synchronize illuminations of the pulse indication light 34 in time with the rate of the pulse measurements. The illuminations provide a visual indication of the pulse rate.

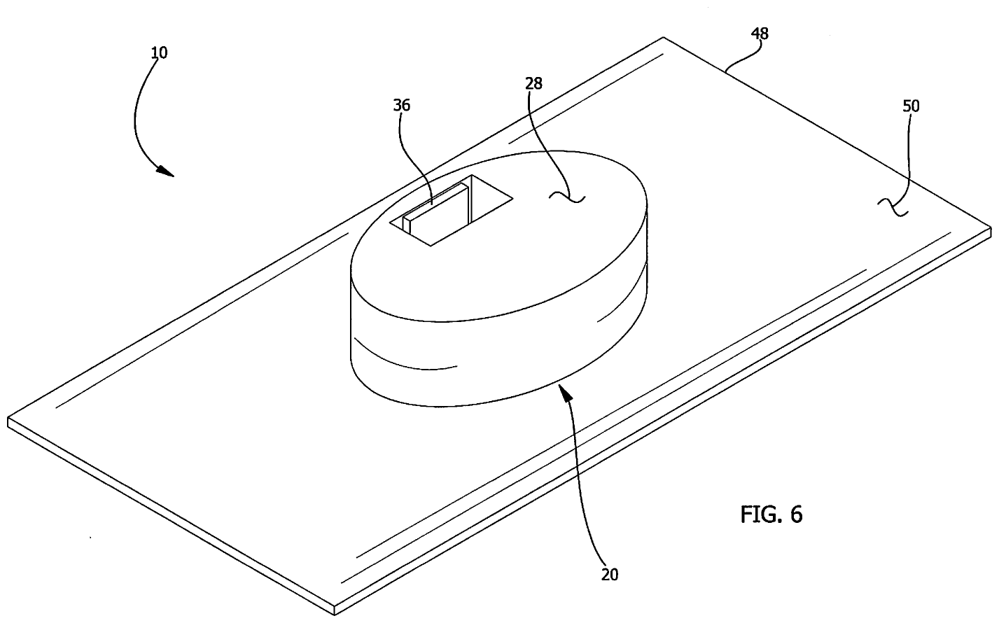

The embodiments depicted in FIGS. 6-8 include a transducer connection port 36. The transducer connection port 36 may be mounted to the perimeter wall 24, for example being positioned on the top wall 28. The transducer connection port 36 is electronically coupled to the central processing unit 30. The transducer connection port 36 is configured to receive a transducer cord 40 that is in communication with a transducer 42 of a photoplethysmogram display screen 44. The transducer cord 40 transmits the measurements of the pulse from the central processing unit 30 to the transducer 42, which converts the measurements into visual indicators of the heart or pulse rate for display on the photoplethysmogram display screen 44. For example, the visual indicators may include wave form graphs of the heart rate or a numerical measurement of the heart rate.

Embodiments such as those depicted in FIGS. 6-8 will generally comprise a heartbeat sensor system 100. For example, the heartbeat sensor device 10 may be used in collaboration with the photoplethysmograph display screen 44, the transducer 42, and the transducer cord 40. The photoplethysmograph display screen 44, the transducer 42, and the transducer cord 40 are generally conventional. The photoplethysmograph display screen 44 is configured to display the heart rate, for example through wave form graphical imagery or numerical indicators. The transducer cord 40 is electronically coupled to the photoplethysmogram display screen 44. The transducer 42 is in communication with the transducer cord 40. The transducer 42 is configured to convert the pulse measured by the photoplethysmogram sensor 46 into the heart rate. The transducer 42 is also in electronic communication with the photoplethysmogram display screen 44. The transducer 42 is configured to transmit the heart rate to the photoplethysmogram display screen 44.

Typically, embodiments will only include one of the pulse indication light 34 and the transducer connection port 36. The visual display of the pulse and heart rate will be provided in real time by either the illuminations of the pulse indication light 34 or the visual displays on the photoplethysmogram display screen 44. These visual indicators can confirm the presence and stability of a pulse in the patient 12. The healthcare professional attending to the patient 12 will not need to stop chest compressions to determine when there is a return of spontaneous circulation in the patient 12. Confirmation of the return of spontaneous circulation will also not be dependent on the manual dexterity or subjective assessment of the healthcare professional, who may have trouble finding the pulse by feel if the pulse is weak or intermittent. The pulse check can therefore be performed with increased objectivity and accuracy over the prior art.

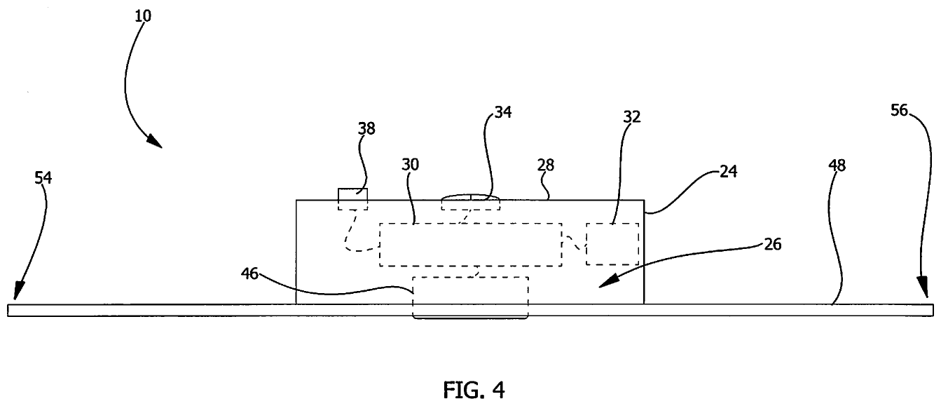

Some embodiments may include a switch 38 that is electronically coupled to the central processing unit 30. The switch 38 is actuatable to turn the central processing unit 30 on or off, for example to conserve the lifetime of the battery 32. In such embodiments, the switch 38 may be mounted to the housing 20, for example being positioned on the top wall 28. Although the embodiments depicted in FIGS. 4, 6, 7, and 8 include the switch 38, the switch 38 is not a necessary component of all embodiments. For example, as shown in FIGS. 1, 5, and 6, the switch 38 may be excluded to increase the ease and quickness of use of the heartbeat sensor device 10.

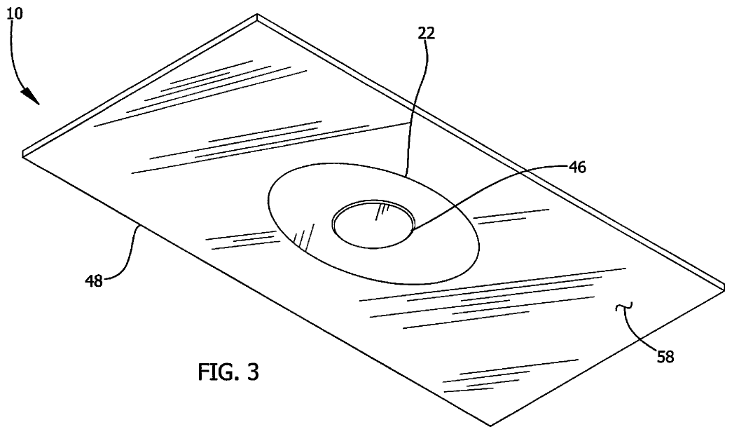

A band 48 may also be attached to the housing 20, extending outwardly from the housing 20. The band 48 has a top side 50 and a bottom side 52. As shown best in FIG. 3, the bottom side 52 is generally aligned with the bottom wall 22 of the housing. The band 48 may also include a first end 54 and a second end 56, with the housing 20 being positioned between the first 54 and second 56 ends. For example, the housing 20 may be approximately half-way between the first 54 and second 56 ends. The band 48 is generally configured to secure the housing 20 to the patient 12. For example, the band 48 may include an adhesive gel pad 58 covering the bottom side 52. The adhesive gel pad 58 may be latex free, such as a medical grade adhesive that can securely and gently adhere the band 48 to the skin of the patient 12.

A cover 60 may be removably affixed to the adhesive gel pad 58. The cover 60 is generally configured to protect the adhesive gel pad 58 from being damaged before use, and to maintain the sterility of the adhesive gel pad 58. The cover 60 may extend across the bottom side 52 and the bottom wall 22 when the cover 60 is affixed to the adhesive gel pad 58. The cover 60 may be easy to peel off of the adhesive gel pad 58 when the heartbeat sensor device 10 is being used. Because typical embodiments are intended for single use, the cover 60 does not need to be replaceable on the adhesive gel pad 58 after use.

In use, the cover 60 is removed from the adhesive gel pad 58 and the heartbeat sensor device 10 is placed on the skin of the patient 12. For example, the heartbeat sensor device 10 may be placed over the right common carotid artery 14 because the right carotid artery 14 is slightly longer than the left carotid artery. The photoplethysmogram sensor 46 can detect changes in the pulse wave over the carotid artery 14 by measuring the pulse. The measurements are communicated to the central processing unit 30. In embodiments including a pulse indication light 34, the central processing unit 30 can illuminate the pulse indication light 34 at the same, real-time rate of the pulse. In embodiments including a transducer connection port 36, the central processing unit 30 can transmit the measurements of the pulse to the transducer 42 through the transducer cord 40 for visual display on the photoplethysmogram display screen 44. The heartbeat sensor device 10 is quick and easy to apply to the patient 12, meaning that the attending healthcare professional will not need to take significant pauses in chest compressions for a pulse check. Once the return of spontaneous circulation is confirmed, the attending healthcare professional can cease chest compressions and continue with the next steps in patient care.

As stated above, embodiments will be relatively small to fit comfortably over the carotid artery 14. For example, embodiments of the heartbeat sensor device 10 may have a height ranging from 8 centimeters to 12 centimeters and a width ranging from 4 centimeters to 8 centimeters.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A heartbeat sensor device comprising:
a housing having a bottom wall and a perimeter wall being attached to and extending upwardly from the bottom wall, the perimeter and bottom walls bounding an interior space;
a central processing unit being positioned within the interior space;
a battery being electronically coupled to the central processing unit, the battery being positioned within the interior space;
a pulse indication light being positioned within the interior space, the pulse indication light being directed outwardly away from the perimeter wall, the pulse indication light being electronically coupled to the central processing unit;
a photoplethysmograph sensor being positioned within the interior space, the photoplethysmograph sensor and being directed outwardly away from the bottom wall, the photoplethysmograph sensor being electronically coupled to the central processing unit;
the photoplethysmography sensor being configured to measure a pulse;
the central processing unit being configured to synchronously illuminate the pulse indication light with the pulse; and
a band being attached to the housing, the band having a top side and a bottom side, the bottom side being aligned with the bottom wall.

2. The heartbeat sensor device of claim 1, wherein the central processing unit is mounted to the housing.

3. The heartbeat sensor device of claim 1, wherein the battery is mounted to the housing.

4. The heartbeat sensor device of claim 1, wherein the pulse indication light is mounted to the housing.

5. The heartbeat sensor device of claim 1, wherein the photoplethysmography sensor is mounted to the housing.

6. The heartbeat sensor device of claim 1, further comprising a switch being electronically coupled to the central processing unit, the switch being actuatable to turn the central processing unit on or off, the switch being mounted to the housing.

7. The heartbeat sensor device of claim 1, further comprising a transducer connection port being positioned on the perimeter wall, the transducer connection port being electronically coupled to the central processing unit, the transducer connection port being configured to receive a transducer cord.

8. The heartbeat sensor device of claim 1, further comprising an adhesive gel pad covering the bottom side.

9. The heartbeat sensor device of claim 8, wherein the adhesive gel pad is latex free.

10. The heartbeat sensor device of claim 9, further comprising a cover being removably affixed to the adhesive gel pad.

11. The heartbeat sensor device of claim 10, wherein the cover extends across the bottom side and the bottom wall when the cover is affixed to the adhesive gel pad.

12. The heartbeat sensor device of claim 1, the band further comprising a first end and a second end.

13. The heartbeat sensor device of claim 12, wherein the housing is positioned at a midsection between the first end and the second end.

14. The heartbeat sensor device of claim 1, the pulse indication light further comprising a light emitting diode.

15. The heartbeat sensor device of claim 1, wherein the pulse indication light is controlled by the central processing unit.

16. A heartbeat sensor device comprising:
a housing having a bottom wall and a perimeter wall being attached to and extending upwardly from the bottom wall, the perimeter and bottom walls bounding an interior space, the perimeter wall having a top wall, the top wall being opposite the bottom wall;
a central processing unit being mounted to the housing, the central processing unit being positioned within the interior space;
a battery being electronically coupled to the central processing unit, the battery being mounted to the housing, the battery being positioned within the interior space;
a photoplethysmograph sensor being mounted to the housing and being directed outwardly away from the bottom wall, the photoplethysmograph sensor being electronically coupled to the central processing unit;
a pulse indication light being electronically coupled to the central processing unit, the pulse indication light being mounted to the housing, the pulse indication light being directed outwardly away from the perimeter wall, the pulse indication light being positioned on the top wall, the pulse indication light being controlled by the central processing unit, the pulse indication light being a light emitting diode;
a switch being electronically coupled to the central processing unit, the switch being actuatable to turn the central processing unit on or off, the switch being mounted to the housing, the switch being positioned on the top wall;
the photoplethysmography sensor being configured to measure a pulse;
the central processing unit being configured to synchronously illuminate the pulse indication light with the pulse;
a band being attached to and extending outwardly from the housing, the band having a top side and a bottom side, the bottom side being aligned with the bottom wall;
the band having a first end and a second end, the housing being positioned between the first end and the second end;
an adhesive gel pad covering the bottom side, the adhesive gel pad being latex free; and
a cover being removably affixed to the adhesive gel pad, the cover extending across the bottom side and the bottom wall when the cover is affixed to the adhesive gel pad.

17. A heartbeat sensor system comprising:
a photoplethysmogram display screen being configured to display a heart rate;
a transducer cord being electronically coupled to the photoplethysmogram display screen;
a transducer being in communication with the transducer cord, the transducer being configured to convert a measurement of a pulse to the heart rate, the transducer being in electronic communication with the photoplethysmogram display screen, the transducer being configured to transmit the heart rate to the photoplethysmogram display screen;
a heartbeat sensor device being electronically coupled to the transducer cord, the heartbeat sensor device including:
a housing having a bottom wall and a perimeter wall being attached to and extending upwardly from the bottom wall, the perimeter and bottom walls bounding an interior space;
a central processing unit being positioned within the interior space;
a battery being electronically coupled to the central processing unit, the battery being positioned within the interior space;
a transducer connection port being electronically coupled to the central processing unit, the transducer connection port being configured to receive the transducer cord;
a photoplethysmograph sensor being positioned within the interior space, the photoplethysmograph sensor and being directed outwardly away from the bottom wall, the photoplethysmograph sensor being electronically coupled to the central processing unit;
the photoplethysmography sensor being configured to measure a pulse, the photoplethysmograph sensor being in communication with the central processing unit to provide the measurement of the pulse to the central processing unit;
the transducer cord being in communication with the central processing unit, the transducer cord being configured to receive the pulse from the central processing unit, the transducer cord configured to transmit the measurement of the pulse to the transducer; and
an adhesive band being attached to the housing, the adhesive band having a top side and a bottom side, the bottom side being aligned with the bottom wall.
